(19) United States
(12) Patent Application Publication
(10) Pub. No.: US 2017/0265801 A1
(43) Pub. Date: Sep. 21, 2017

(54) Bruxism Detection System With Chin-Mounted Accelerometer Sensor

(71) Applicants: Aalok Nital Patwa, San Jose, CA (US); Manasi Nital Patwa, San Jose, CA (US)

(72) Inventors: Aalok Nital Patwa, San Jose, CA (US); Manasi Nital Patwa, San Jose, CA (US)

(21) Appl. No.: 15/459,490

(22) Filed: Mar. 15, 2017

Related U.S. Application Data
(60) Provisional application No. 62/308,847, filed on Mar. 15, 2016.

(51) Int. Cl. A61B 5/00; A61B 5/11

(52) U.S. Cl. CPC A61B 5/4557; A61B 5/11; A61B 5/7282; A61B 5/6814; A61B 5/6831; A61B 5/0022; A61B 2562/0219

(57) ABSTRACT

A bruxism detection system is provided that includes a chin-mounted acceleration sensor for detection of teeth grinding and teeth tapping. The system generally includes an acceleration sensor that is adapted to be removably and externally mounted with respect to an individual's chin, a bruxism recording and processing system operable on a local processor, and bruxism analysis software that is operable on the local processor or an adjoint processor (or a combination thereof). The system is designed to be used in any convenient location, including an individual's home, and is generally reusable by multiple people, thus reducing the cost of bruxism diagnosis and bringing a reliable and effective diagnosis tool to the general public.

### CROSS-REFERENCE TO RELATED APPLICATION

The present application claims priority benefit to a provisional patent application entitled “A Bruxism Detection System with Chin-Mounted Accelerometer Sensor,” which was filed on Mar. 15, 2016, and assigned Ser. No. 62/308,847. The entire content of the foregoing provisional patent application is incorporated herein by reference.

### BACKGROUND

1. Technical Field

A bruxism detection system is provided that includes a chin-mounted acceleration sensor for detection of teeth grinding and teeth tapping. The system generally includes an acceleration sensor that is adapted to be removably and externally mounted with respect to an individual's chin, a bruxism recording and processing system operable on a local processor, and bruxism analysis software that is operable on the local processor or an adjoint processor (or a combination thereof). The system is designed to be used in any convenient location, including an individual's home, and is generally reusable by multiple people, thus reducing the cost of bruxism diagnosis and bringing a reliable and effective diagnosis tool to the general public.

2. Background Art

Bruxism is a common disorder. Reports of prevalence range from 8-31% in the general population. Bruxism is manifested by teeth grinding, i.e., teeth sliding back and forth over each other, or jaw clenching, i.e., tightly holding top and bottom teeth together, or a combination of both teeth grinding and jaw clenching. Teeth grinding results in excessive wear and tear on teeth; causing worn enamel, eroded dentin, increased teeth sensitivity, teeth cracks and teeth fractures. Jaw clenching results in pain in jaw muscles, headaches and Masseter hypertrophy. Jaw clenching is also bad for teeth because of excessive pressure applied to teeth. It is believed that increased stress levels result in increased bruxism.

Bruxism can happen either while an individual is awake (“waking bruxism”) or while an individual is sleeping (“sleeping bruxism”). Waking bruxism is more commonly characterized by jaw clenching. Sleeping bruxism is more commonly characterized by teeth grinding and may also be accompanied by jaw clenching.

Sleep bruxism is a difficult condition to detect. Teeth grinding may produce sounds, but since the person is sleeping, someone else has to be awake to hear the sounds. Jaw clenching will generally result in muscle pain or headache, but the person may not report it. Hence, in a majority of cases, Sleep bruxism is detected only when a patient's dentist observes uneven wear of tooth enamel, dentin or a cracked tooth. By this time, teeth are already damaged and the opportunity to preemptively address the bruxism condition has been missed.

As is apparent, there is a significant benefit of designing a system that can detect sleep bruxism early on, and particularly before it causes permanent teeth damage.

Conventionally, systems designed to detect sleep bruxism have employed jaw-mounted surface electromyographic (EMG) sensors. EMG sensors detect the electrical activity produced by muscles. The effectiveness of EMG sensors for bruxism diagnosis may be limited because detection is affected by skin resistance. To avoid potentially unreliable measurements, the skin surface must be cleaned of dead cells, dirt and natural oil to reduce the skin resistance. EMG sensors also require carefully tuned amplifier and more sophisticated processing software. In addition, EMG sensors cannot accurately differentiate teeth clenching against teeth grinding against simply tapping the teeth.

Other known systems for detection of sleep bruxism are intraoral devices with pressure sensors. The bite forces are detected by the pressure sensors mounted on teeth. All intraoral devices face serious challenges of safety, size and power consumption. As a result, use of intraoral devices for bruxism detection are likely to be severely limited.

Publications of background relevance to the systems and methods of the present disclosure are U.S. Pat. No. 7,616,988 to Stahmann et al., U.S. Pat. No. 8,544,322 to Minami et al., U.S. Pat. No. 8,657,756 to Stahmann et al., U.S. Pat. No. 8,790,264 to Sandler et al., US Patent Publn. No. 2015/0335288 to Toth et al., and US Patent Publn. No. 2017/0035350 to Alessie, as well as a publication entitled “Identification of the occurrence and pattern of masseter muscle activities during sleep using EMG and accelerometer systems,” by H. Yoshimi et al. (Head & Face Medicine, 2009, 5:7, Feb. 11, 2009).

However, despite efforts to date, a need remains for a bruxism detection system that is easy to use, provides reliable bruxism detection, and supports clinical and operational requirements so as to manage energy-related constraints. These and other objectives are satisfied by the bruxism detection system described herein.

### SUMMARY

A bruxism detection system is provided that includes a chin-mounted acceleration sensor (i.e., accelerometer) for detection of teeth grinding and teeth tapping. Although the present disclosure is provided with specific reference to bruxism diagnostic applications, it is to be understood that the systems and methods described herein may find application in other anatomical/clinical settings, e.g., for detection/diagnosis of restless leg syndrome or the like. Thus, persons skilled in the art will understand from the description provided herein that motion detection and analysis may be implemented with respect to other body parts/locations for desired clinical purposes.

The disclosed bruxism detection system generally includes an acceleration sensor that may be detachably mounted with respect to an individual's chin. The acceleration sensor is incorporated into a module that further includes an energy source, e.g., a battery, local processing system/memory, and communication functionality. The acceleration sensor-containing module generally communicates with a remote or adjoint processing unit, e.g., a smartphone or other mobile device (e.g., a personal digital assistant), a laptop or desktop computer, and/or a networked system, e.g., the Internet, that allows communication with a hosted website or the like. In short, the disclosed acceleration sensor-containing module is configured and programmed to sense and collect measurements and data relevant to bruxism diagnosis, to store those measurements/data, and to communicate those measurements/data to a remote/adjoint electronic system for further processing and display.

The local processor may be further programmed to determine whether the measurements/data generated by the accelerometer correspond to a bruxism condition, either alone or in combination with the remote/adjoint electronic system. The communication of the measurements/data may be by wired communication, wireless communication or a combination thereof.

Thus, the disclosed bruxism detection system includes bruxism recording and processing functionalities that generally operate locally within the acceleration sensor-containing module, and bruxism analysis software that is adapted to operate on a processing unit independent o the acceleration sensor-containing module, thereby limiting energy/battery use in the module. The disclosed bruxism detection system supports significant flexibility in operational implementation in that the bruxism analysis software may operate, in whole or in part, on the local processing unit contained in the module, in the remote/adjoint processing unit, or cooperatively across both processors. Communication of the measurement/data from the acceleration sensor-containing module to the independent/adjoint processing unit is generally controlled so as to limit energy/battery requirements, e.g., based on burst transmissions of data that has been pre-filtered within the module so as to limit transmission to measurements/data that is relevant to operations of bruxism analysis software and/or display functionalities associated with the remote/adjoint processor.

The disclosed bruxism detection system has wide ranging use and applicability. The acceleration sensor-containing module is designed for patient use, i.e., a health care professional is not required, and its use is indicated for any convenient location, including an individual's home. The disclosed acceleration sensor-containing module and associated chin-mounting system are generally reusable by multiple people, thus reducing the cost of bruxism diagnosis and bringing a reliable and effective diagnosis tool to the general public.

### BRIEF DESCRIPTION OF THE FIGURES

To assist those of skill in the art in making and using the disclosed bruxism detection system, reference is made to the accompanying figures, wherein:

FIG. 1 is a schematic depiction of an individual's face with an exemplary bruxism detection module detachably mounted with respect to the individual's chin;

FIG. 2 is a schematic depiction of data collection and communication according to an exemplary embodiment of the disclosed bruxism detection system;

FIG. 3 is an exemplary flowchart depicting the processing of accelerometer measurements to assess the presence of bruxism according to the present disclosure;

FIG. 4 is an exemplary evaluative technique for three sets of acceleration data according to the detection system of the present disclosure; and

FIG. 5 is a tabular presentation of bruxism analysis with respect to the acceleration data set forth in FIG. 4.

### DESCRIPTION OF EXEMPLARY EMBODIMENT(S)

The disclosed bruxism detection system uses an accelerometer sensor that is adapted to be detachably mounted relative to an individual's chin to sense and collect motion data in three axes, i.e., the x-axis, the y-axis and the z-axis. The motion data is initially stored on local hardware associated with the module that contains the acceleration sensor. Since an individual's chin has the maximum mandibular acceleration during episodes of teeth grinding or teeth clenching, the best mounting position for accelerometer sensor is the chin.

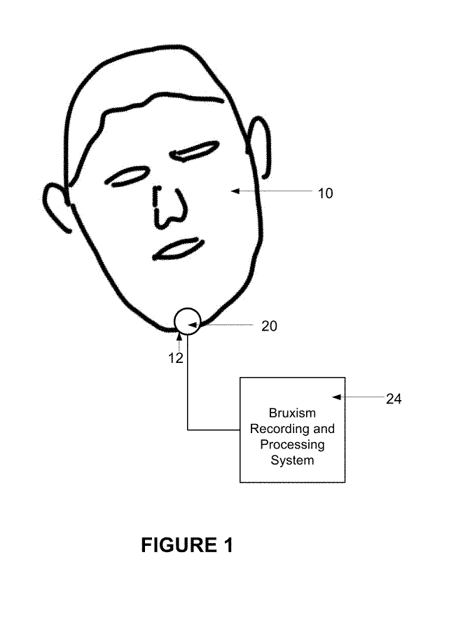

With reference to FIG. 1 which schematically depicts an individual's face 10, a module 20 is detachably mounted with respect to chin 12 of the individual's face 10. Module 20 contains, inter alia, an acceleration sensor 22 that is adapted to sense motion in three axes, i.e., the x-axis, the y-axis and the z-axis. Mounting of module 20 relative to chin 12 is generally accomplished with an anti-allergy material, such as hospital-grade cloth tape with a protective gauze pad. Alternative mounting mechanisms may be employed, as will be readily apparent to persons skilled in the art, e.g., using strap(s), suitably sized bandage(s), adhesives or the like. This method of mounting of module 20 relative to an individual's chin advantageously permits reuse of the acceleration sensor 22 and electronics associated with module 20.

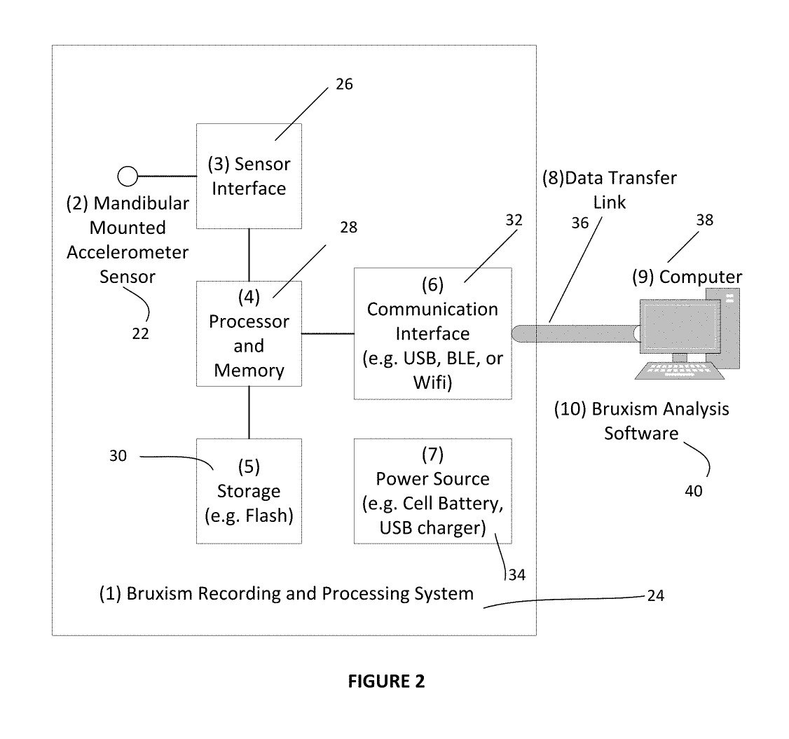

The acceleration sensor or accelerometer 22 is connected to and in communication with a sensor interface of the disclosed bruxism recording and processing system 24 operating on a local processor contained within module 20. Thus, with reference to FIGS. 1 and 2, the bruxism recording and processing system 24 contains or includes (i) a sensor interface 26, (ii) a processor with memory 28, (iii) storage 30, (iv) a communication interface 32, and (v) a power source 34. Storage 30 can be any non-volatile storage, such as flash memory. Communication interface 32 can be any wired interface, such as USB or Ethernet, or any wireless interface, such as Bluetooth, Bluetooth-Low-Energy(BLE), Wifi or Zigbee. The power source 34 can be a battery or a USB charger.

In use, an individual affixes module 20 to his/her chin and goes to sleep with accelerometer 22 (which is within module 20) positioned to sense movements of the chin 12. The bruxism recording and processing system 24 (associated with local hardware/processor also within module 20) is connected to and in communication with accelerometer 22. As soon as the bruxism recording and processing system 24 is turned on, the system locally acquires, processes and stores the accelerometer data.

More particularly, the bruxism recording and processing system 24 is advantageously programmed to minimize energy use, thereby extending battery life and ensuring sufficient power for overnight use of the disclosed bruxism detection system. Thus, in exemplary embodiments of the present disclosure, the bruxism recording and processing system 24 pre-processes motion-related measurements/data, but does not automatically activate/initiate its communication interface 32 to wirelessly transmit such information to a remote electronic device. The pre-processing of the bruxism recording and processing system 24 filters out data that is not relevant to bruxism detection, and typically transmits in burst transmissions relevant data that satisfies a threshold content level to a remote processing unit. To effectuate such transmission, the bruxism recording and processing system 24 and the remote/adjoint device engage in an electronic handshake to confirm/establish that applicable transmission parameters are satisfied.

When transmissions are to be undertaken, the bruxism recording and processing system 24 engages in communication with a remote/adjoint electronic device via the communication interface 32 which forms a data transfer link 36 with the remote/adjoint electronic device, e.g., computer 38. Computer 38 may be in close proximity to the individual undergoing the bruxism testing, e.g., a smartphone, laptop, desktop, etc., or may be remotely located and in communication with the bruxism recording and processing system 24 through conventional network-based communication systems, e.g., by way of a modem to processor(s) that operate remotely, e.g., in the “cloud”. Thus, the pre-processed accelerometer data is sent to the computer 38, wherever located and in whatever hardware form, via the data transfer link 36.

The adjoint computer 38 is generally programmed to run bruxism analysis software 40 according to the present disclosure. However, significant flexibility is provided by the present disclosure in that the disclosed bruxism analysis software may operate, in whole or in part, on the local processor positioned within module 20 as well. Thus, the required processing capability for bruxism analysis may be deployed in one or both hardware locations (i.e., local and adjoint processor), and may be operated in one or both hardware locations (i.e., local and adjoint processor), as desired by the user (or dictated by other factors, such as energy/power requirements and availability). Of note, the adjoint computer 38 is generally programmed to provide visualization/display and reporting functionalities for an end user interacting with the adjoint computer 38.

The bruxism analysis software 40 functions to analyze the processed accelerometer data, assess whether the accelerometer data corresponds to behaviors consistent with bruxism, and generate reports regarding the existence of bruxism. The bruxism analysis can be done in real-time mode or off-line mode. In the real-time mode, the accelerometer data is generally acquired, processed, stored and sent over the data transfer link by the local processor to the adjoint computer 38 where the bruxism analysis software 40 is running. In the off-line mode, the accelerometer data is acquired, processed and stored while the patient is sleeping. When the patient wakes up (or whenever convenient), the stored accelerometer data may be transferred over the data link 36 to the adjoint computer 38, at which time it is analyzed by the bruxism analysis software 40 running on the adjoint computer 38.

The system of the present disclosure detects teeth grinding, teeth tapping and teeth clenching by means of detecting, measuring and processing mandibular motion. The accelerometer sensor 22 included in the module 20 measures acceleration. When the mandibular motion starts, acceleration is recorded. Teeth grinding and teeth tapping both have rapid back-and-forth mandibular motion. So the accelerometer 22 senses and records a time-varying acceleration.

Clenching has a motion at the beginning and then at the end. So the accelerometer 22 senses and records acceleration change at the beginning of clenching and at the end of clenching.

The bruxism motion has “signature” motion-related characteristics which are characterized by parameters such as mean of the readings, variance of the readings, #of mean crossings, average time to cross the mean, frequency components of motion and shape of the waveform. By examining the acceleration readings and comparing them against known ‘signature’ of different types of bruxism conditions, the analysis software can detect bruxism.

During sleep there are other motions unrelated to bruxism. Turning the head, changing the sleeping position and swallowing are quite common. Sleep apnea and acid reflux are among the clinical conditions that can result in motion in the mandibular region, particularly at the chin. Each of these unrelated motions has its own ‘signature’. The analysis software of the present disclosure is programmed to recognize these ‘signatures’ and to reject the motions unrelated to bruxism.

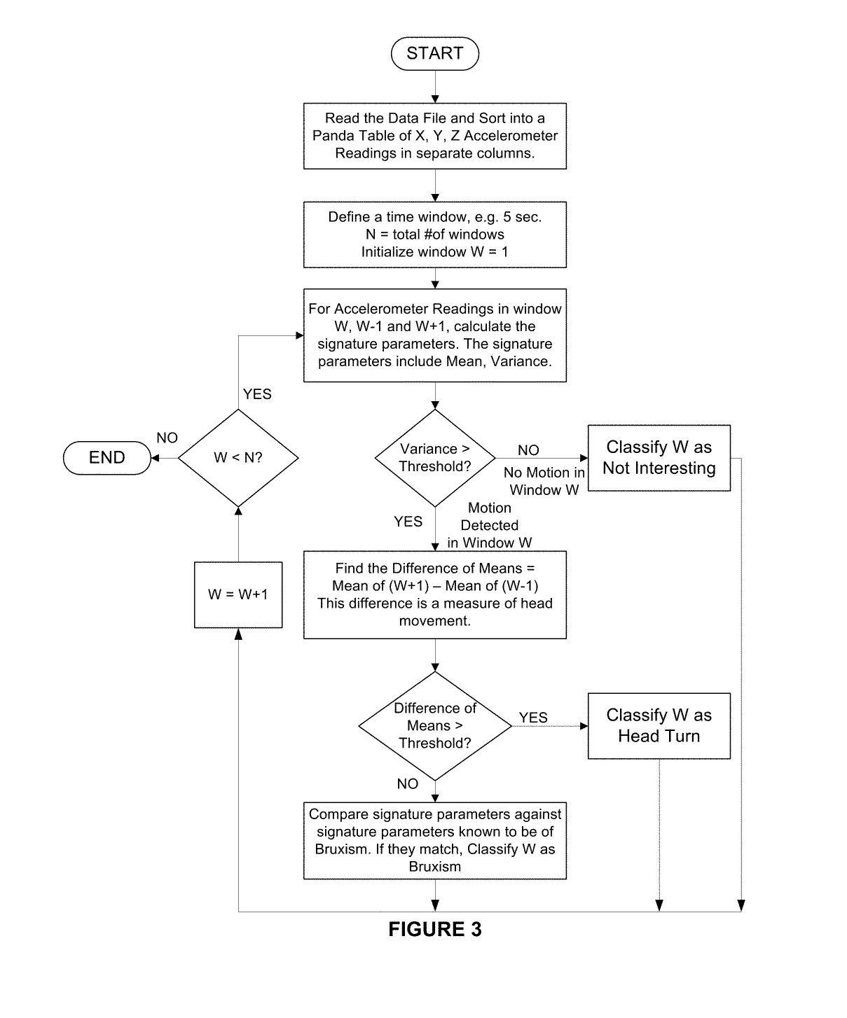

FIG. 3 illustrates a flow chart of exemplary operation of bruxism analysis software 40 according to the present disclosure for detection of bruxism and rejection of unrelated motions, e.g., head turns/sleeping position change. The detection is based on deriving signature parameters of current time window and comparing it against known signature parameters of bruxism. The signature parameters include, but are not limited to, mean, variance, #of mean-crossings, average time to cross the mean, frequency components of motion, and shape of the waveform.

As shown in FIG. 3, bruxism analysis according to the present disclosure may advantageously include sorting the accelerometer data into separate columns/fields based on the axis of the measured motion, i.e., x-axis, y-axis and z-axis. A time window for data evaluation is defined, e.g., 5 seconds, and then determine whether motion is reflected in the time window. To the extent no motion is found in the window, the window is classified as “not interesting” and, depending on how many windows have been reviewed, another time window may be sampled. To the extent motion is found in the time window, the mean movement, frequency component of motion and variance movement values are compared to threshold values to determine whether the movement values correspond to parameters consistent with actions distinct from bruxism, e.g., head movement. To the extent the movement values correspond to parameters consistent with bruxism, the noted time window is classified as indicative of bruxism.

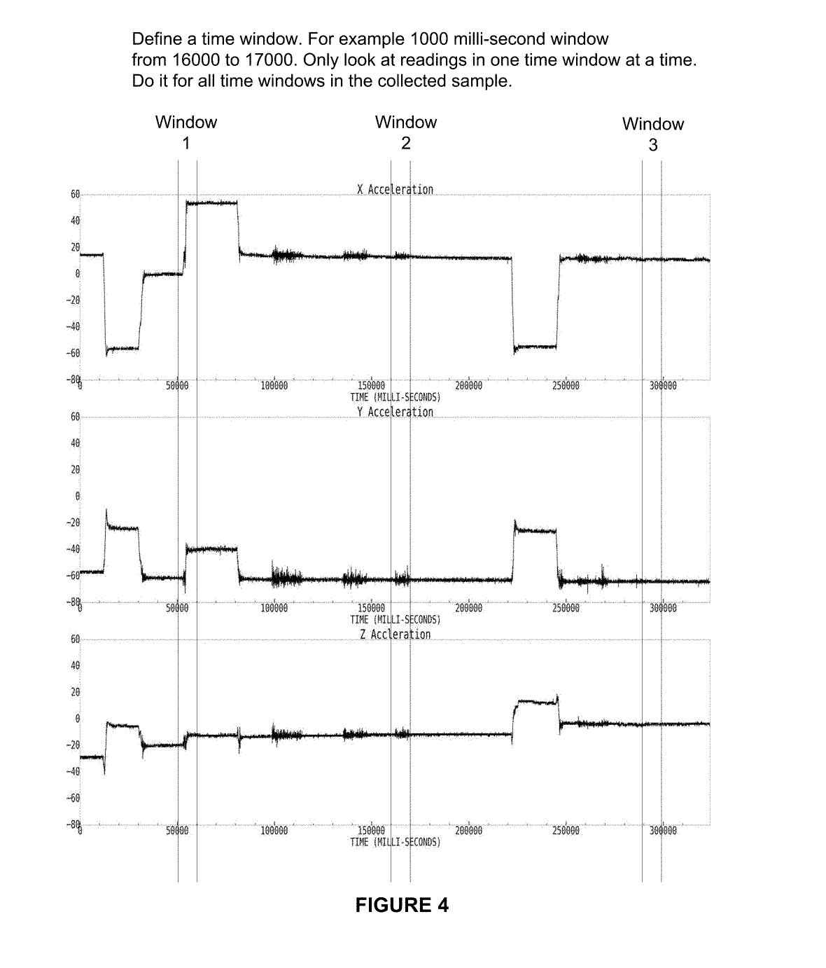

FIG. 4 shows exemplary accelerometer readings for simulated teeth grinding/teeth tapping and head turning.

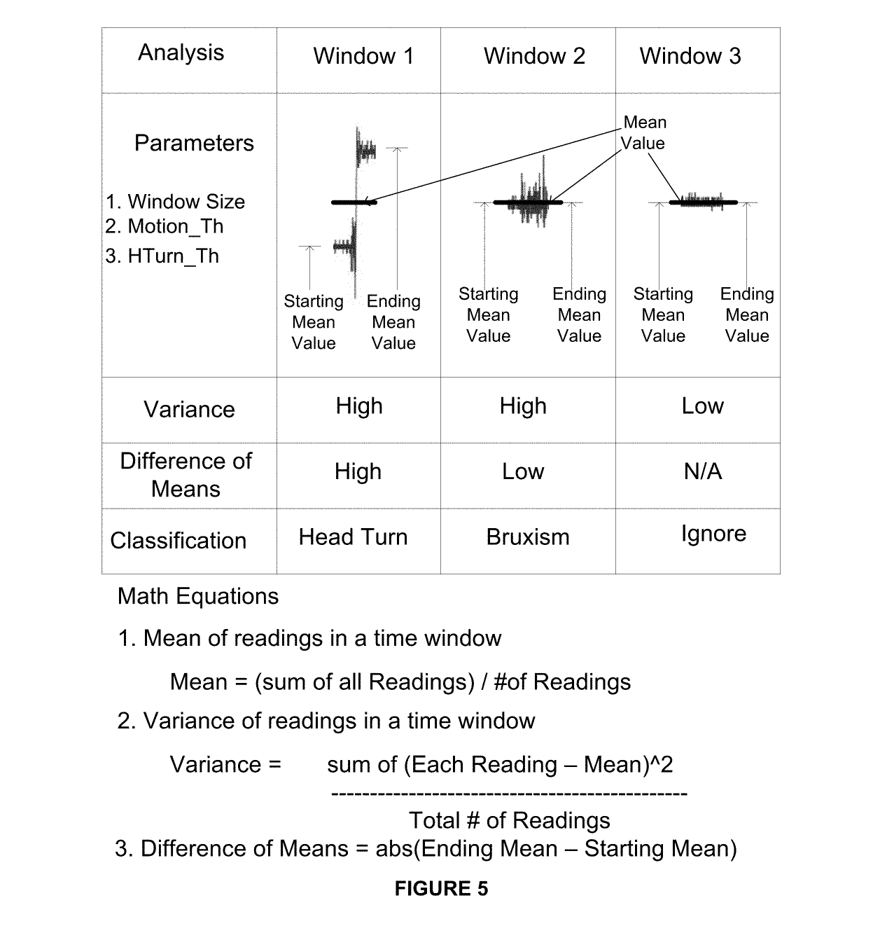

FIG. 5 shows in tabular form how the bruxism analysis software 40 of the present disclosure processes the accelerometer readings set forth in FIG. 4 to identify/report bruxism events and reject head turn events. As described herein, the bruxism determination is made based on accelerometer data from a single accelerometer that is mounted with respect to an individual's chin using one or more of the following signature parameters, namely mean, variance and frequency components of motion.

As described herein, the disclosed bruxism detection system works better than EMG-based sensor systems for teeth grinding and teeth tapping because the motion detection signal level available from an accelerometer sensor is stronger than the electrical activity signal level available from EMG sensors. In addition, the disclosed bruxism detection system is better than an intra oral system because the accelerometer sensor is mounted on face and thus none of the issues of an intraoral system—safety, size, power consumption—arise. Still further, accelerometer sensors are less expensive than EMG sensors as they are used in high volume in many products, such as smart phones, gaming controllers, laptops, airbags, so the cost of our bruxism detection system is lower. Also, all the components of the device are reusable. Thus, the disclosed bruxism detection system offers many advantages and brings at-home, low-cost bruxism diagnosis to entire population without the need for health care professional intervention or involvement.

Although the present disclosure has been described with reference to exemplary embodiments and implementations, the present disclosure is not limited by or to such exemplary embodiments/implementations. Rather, the disclosed bruxism detection system is susceptible to various modifications, refinements and/or enhancements without departing from the spirit or scope of the present invention.

1. A bruxism detection system, comprising:
a. a detection module that includes a single accelerometer and a local processor that includes a recording and processing system, the recording and processing system including (i) a sensor interface, (ii) a processor with memory, (iii) non-volatile storage, (iv) a communication interface, and (v) a power source; and
b. an adjoint processor in communication with the recording and processing system of the detection module,
wherein at least one of the local processor and the adjoint processor is programmed to detect bruxism from multi-axis motion data collected by the single accelerometer by calculating motion signature parameters that include one or more of mean, variance and frequency components of motion, and comparing the calculated motion signature parameters with preconfigured motion signature parameters that correspond to bruxism.

2. The bruxism detection system of claim 1, further comprising a mechanism for detachably securing the detection module to a chin of an individual.

3. The bruxism detection system of claim 2, wherein the mechanism is selected from the group consisting of one or more cloth tapes, gauze pads, straps, bandages and combinations thereof.

4. The bruxism detection system of claim 1, wherein the recording and processing system pre-filters motion data captured by the single accelerometer to eliminate data that is not relevant to detection of bruxism.

5. The bruxism detection system of claim 1, wherein the recording and processing system communicates with the adjoint processor in burst transmissions.

6. The bruxism detection system of claim 1, wherein the recording and processing system establishes an electronic handshake with the adjoint processor before communicating motion data captured by the single accelerometer.

7. The bruxism detection system of claim 1, wherein the adjoint processor is a computer.

8. The bruxism detection system of claim 7, wherein the adjoint computer is selected from the group consisting of a smartphone, laptop computer and a desktop computer.

9. The bruxism detection system of claim 1, wherein the adjoint processor is remotely located relative to the detection module and wherein motion data communicated from the recording and processing system is transmitted over a network.

10. The bruxism detection system of claim 1, wherein the local processor is programmed to detect bruxism from multi-axis motion data collected by the single accelerometer by calculating motion signature parameters that include one or more of mean, variance and frequency components of motion, and comparing the calculated motion signature parameters with preconfigured motion signature parameters that correspond to bruxism.

11. The bruxism detection system of claim 1, wherein the adjoint processor is programmed to detect bruxism from multi-axis motion data collected by the single accelerometer by calculating motion signature parameters that include one or more of mean, variance and frequency components of motion, and comparing the calculated motion signature parameters with preconfigured motion signature parameters that correspond to bruxism.

12. The bruxism detection system of claim 1, both the local processor and the adjoint processor are programmed to detect bruxism from multi-axis motion data collected by the single accelerometer by calculating motion signature parameters that include one or more of mean, variance and frequency components of motion, and comparing the calculated motion signature parameters with preconfigured motion signature parameters that correspond to bruxism.

13. A method for detecting bruxism, comprising:
a. detachably mounting a detection module with respect to a chin of an individual, the detection module including a single accelerometer and a local processor that includes a recording and processing system, wherein the recording and processing system includes (i) a sensor interface, (ii) a processor with memory, (iii) non-volatile storage, (iv) a communication interface, and (v) a power source;
b. collecting multi-axis motion data with the accelerometer included in the detection module;
c. pre-filtering the multi-axis motion data to exclude data that is not relevant to bruxism detection; and
d. transmitting the filtered motion data to an adjoint processor; and
e. processing the filtered motion data at the local processor, the adjoint processor or a combination of the local processor and the adjoint processor to detect bruxism, the processing including calculation of motion signature parameters that include one or more of mean, variance and frequency components of motion, and comparison of the calculated motion signature parameters with preconfigured motion signature parameters that correspond to bruxism.
